(19) United States
(12) Patent Application Publication
(10) Pub. No.: US 2006/0205618 A9
(43) Pub. Date: Sep. 14, 2006

(54) Detergent cosmetic compositions comprising at least one polymer chosen from cationic and amphoteric polymers, at least one detergent surfactant, and at least one drawing polymer, and process of use

(76) Inventors: Henri Samain, Bievres (FR); Geraldine Fack, Levallois (FR); Franck Giroud, Clichy (FR); Laurence Paul, Saint Leu La Foret (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 11/030,296

(22) Filed: Jan. 7, 2005

Related U.S. Application Data
(60) Provisional application No. 60/537,912, filed on Jan. 22, 2004.
(60) Provisional application No. 60/538,251, filed on Jan. 23, 2004.

(30) Foreign Application Priority Data
Jan. 7, 2004 (FR) 04 50029
Jan. 7, 2004 (FR) 04 50035

(51) Int. Cl. A61K 7/075

(52) U.S. Cl. 510/119; 510/123

(57) ABSTRACT

The present disclosure relates to novel washing compositions for keratin materials, for instance for the hair, comprising, in a cosmetically acceptable medium, at least one polymer chosen from cationic and amphoteric polymers, at least one detergent surfactant chosen from anionic, nonionic and amphoteric detergent surfactants and at least one drawing polymer with a drawing power of greater than 5 cm. Also disclosed herein is a process for treating keratin materials using the compositions of the present disclosure.

This application claims benefit of U.S. Provisional Application No. 60/537,912, filed Jan. 22, 2004 and U.S. Provisional Application No. 60/538,251, filed Jan. 23, 2004.

The present disclosure relates to novel cosmetic compositions with beneficial properties, intended for cleaning, conditioning and styling keratin materials, such as the hair, and comprising, in a cosmetically acceptable medium, a washing base comprising at least one surfactant with detergent power, at least one polymer chosen from cationic and amphoteric polymers, and at least one drawing polymer of high molecular weight. The present disclosure also relates to the use of the compositions in the abovementioned cosmetic applications.

It is known practice to use detergent hair compositions (or shampoos) based essentially on standard surfactants of anionic, nonionic and/or amphoteric type, for example, such as of anionic type, to clean and/or wash the hair. The compositions can be applied to wet hair and the lather generated by massaging or rubbing with the hands may remove, after rinsing with water, the various types of soiling initially present on the hair.

While these basic compositions may have good washing power, the intrinsic cosmetic properties associated therewith nevertheless can be fairly poor, possibly owing, for instance, to the fact that the relatively aggressive nature of such a cleaning treatment can, in the long run, lead to more or less pronounced damage to the hair fiber. This damage may be associated, for example, with the gradual removal of the lipids or proteins comprised in or on the surface of this fiber.

Thus, in order to improve the cosmetic properties of the above detergent compositions, for instance, those that are intended to be applied to sensitized hair (i.e. hair that has been damaged or made brittle, for example due to the chemical action of atmospheric agents and/or hair treatments such as permanent-waving, dyeing or bleaching), it is now common practice to introduce additional cosmetic agents known as conditioners into these compositions. The conditioners are intended mainly to repair or limit the harmful or undesirable effects induced by the various treatments or aggressions to which the hair fibers may be subjected to more or less repeatedly. These conditioners may, of course, also improve the cosmetic behavior of natural hair.

The conditioners most commonly used to date in shampoos are cationic polymers, which can give washed, dry or wet hair at least one benefit chosen from an ease of disentangling, softness and smoothness that can be markedly better than those that may be obtained with corresponding cleaning compositions from which they are absent.

Moreover, it has for some time been sought to obtain conditioning shampoos that are capable of giving washed hair not only at least one of the cosmetic properties mentioned above but also, to a greater or lesser extent, styling, volume, shaping and hold properties. The washing shampoos with improved general cosmetic properties are often referred to for simplicity as “styling shampoos,” and this term will be adopted in the description hereinbelow.

However, despite the progress made recently in the field of styling shampoos, these shampoos may not be completely satisfactory, and as such there is currently still a strong need as regards being able to provide novel products that give better performance with respect to at least one of the cosmetic properties mentioned above. For example, it can be necessary to use a styling product after shampooing, to give the hair a shape and to fix the style. The present disclosure is directed towards satisfying such a need.

Thus, after considerable research conducted in this matter, it has been found, entirely surprisingly and unexpectedly, that by combining cationic or amphoteric polymers with certain high molecular weight polymers that have a specific drawing power in detergent compositions, it is possible to substantially and significantly improve the styling and hold properties, while at the same time maintaining good intrinsic washing power and cosmetic properties. For example, the compositions can make it possible to obtain very good hold and a certain amount of volume for the hair, i.e., a styling effect similar to that obtained with a fixing styling gel used after shampooing. It moreover can be found that the keratin fibers are strengthened (hardened) as a result of using the disclosed compositions.

Without wishing to limit the present disclosure to any theory, it would seem that there may be specific interactions and/or affinities between the cationic or amphoteric polymers, the high molecular weight water-soluble drawing polymers in accordance with the present disclosure, and the hair, which promote a regular, large and lasting deposition of the high molecular weight water-soluble drawing polymers at the surface of the hair. This qualitative and quantitative deposition may be, for instance, one of the causes of the improvement that can be observed at the level of the final cosmetic properties, such as the ease of styling, the hold, the vitality and the volume of the treated hair. All these discoveries form the basis of the present disclosure.

Accordingly, the present disclosure relates to novel compositions for washing keratin materials, such as the hair, comprising, in a cosmetically acceptable aqueous medium, at least one drawing polymer with a drawing power of greater than 5 cm, at least one polymer chosen from cationic and amphoteric polymers different from the at least one drawing polymer, and, for example, at least 4% of at least one detergent surfactant chosen from anionic, nonionic and amphoteric detergent surfactants.

Another aspect of the present disclosure is also the cosmetic use of the above compositions for cleaning, conditioning, caring for and styling keratin materials, such as the hair and the eyelashes.

Yet another subject of the present disclosure is a cosmetic process for treating keratin materials, such as keratin fibers, using the compositions as disclosed herein, wherein the keratin materials are, for instance, the hair and the eyelashes.

In one embodiment of the present disclosure, the composition as disclosed herein is a shampoo.

However, other characteristics, aspects and benefits of the present disclosure will emerge more clearly upon reading the description that follows, and also the concrete, but in no way limiting, examples intended to illustrate it.

As used herein, the term “drawing power of a polymer” is understood to mean the length of the polymer yarn obtained at the breaking point of the yarn according to the procedure defined below.

The drawing power of the polymers that may be used as disclosed herein is the power measured for a composition comprising (% by weight):

| | |
|---|---|
| Sodium lauryl ether sulphate oxyethylenated | 12.5% |
| with 2.2 mol of ethylene oxide | |
| Cocoamidopropylbetaine | 2.5% |
| Test polymer | 1% |
| Water | qs 100% |

The drawing power is measured using a TA-TX2 texture analyser (Rheo/stable Micro Systems). The measurement is performed after compression of the product:
Displacement of the disc (35 mm aluminium cylinder) at a speed of 2.5 mm/s and detection of the compression strength
penetration into the product at the same speed to a depth of 10 mm
removal of the probe at a speed of 2.5 mm/s
measurement of the displacement of the probe and detection of the breaking point of the product yarn.

In the present disclosure, the polymers with a drawing power of greater than 5 cm will also be referred to as drawing polymers.

The at least one polymer with a drawing power of greater than 5 cm (drawing polymer) may be chosen from, for example, either (a1) a dispersion of particles of at least one water-soluble polymer with a weight-average molecular mass of greater than 10⁶ in a saline aqueous solution, obtained by heterogeneous free-radical polymerization of water-soluble monomers with precipitation of the polymer formed, with the proviso that at least one of the monomers is cationic, or (a2) an aqueous solution of at least one water-soluble polymer with a weight-average molecular mass of greater than 10⁶, obtained by heterogeneous free-radical polymerization of water-soluble monomers with precipitation of the polymer formed, with the proviso that at least one of the monomers is cationic.

The at least one high molecular weight water-soluble drawing polymer as defined above can be chosen from cationic and amphoteric polyelectrolytes, i.e., a polyelectrolyte polymerized using at least one cationic monomer of formula (I). For example, the at least one high molecular weight water soluble drawing polymer can be cationic. As used herein, the term “cationic polymer” is understood to mean polymers comprising cationic monomers and possibly nonionic monomers.

As indicated above, the synthesis of the at least one high molecular weight water-soluble drawing polymer, as used herein, takes place by heterogeneous free-radical polymerization of water-soluble monomers comprising at least one ethylenic unsaturation. The polymerization takes place in an aqueous solution of a mineral electrolyte (salt) having an ionic strength that is sufficient to cause precipitation of the polymer formed as soon as it has reached a certain molecular mass. This polymerization technique thus allows, by virtue of the well-known phenomenon of salting out, the preparation of saline aqueous dispersions of water-soluble polymer particles. The polymers thus synthesized are distinguished by a high weight-average molecular mass, which is greater than 10⁶.

The technique of heterogeneous free-radical polymerization in an aqueous medium with precipitation of the polymer formed is described, for example, in U.S. Pat. No. 4,929,655, in European Patent Application No. EP 0 943 628 or in International Patent Application No. WO 02/34796.

To ensure the stability of the dispersions of polymer particles during the synthesis and during storage, it may be desired to perform the polymerization in the presence of a dispersant. The dispersant can be, for example, a polyelectrolyte, which, unlike the high molecular weight polymer used as disclosed herein, is soluble in the aqueous polymerization medium of high ionic strength. The dispersing polyelectrolyte can have, for example, a charge identical to that of the polymer synthesized. In other words, for the synthesis of cationic polyelectrolytes, a cationic dispersing polyelectrolyte can be used.

Non-limiting examples of dispersants that may be mentioned include the cationic polyelectrolytes obtained by polymerization of from 50 mol % to 100 mol % of at least one cationic monomer chosen from the salts, such as the hydrochlorides or sulphates, of dimethylaminoethyl (meth)acrylate, of N-dimethylaminopropyl(meth)-acrylamide or of di(meth)allylamine, (meth)acryloyloxy-ethyltrimethylammonium chloride, (meth)acrylamido-propyltrimethylammonium chloride and dimethyldiallyl-ammonium chloride, and of from 50 mol % to 0 mol % of acrylamide. A polyamine such as a polyalkyleneamine may also be used.

The at least one dispersant can be present, for example, in an amount ranging from 1% to 10% by weight, relative to the total weight of the monomers to be polymerized.

The saline aqueous solution that serves as synthesis and dispersion medium for the at least one high molecular weight water-soluble drawing polymer is a solution of at least one mineral salt, for instance, chosen from divalent anionic salts. Non-limiting examples of anionic salts that may be mentioned include ammonium sulphate, ammonium hydrogen sulphate, sodium sulphate, sodium hydrogen sulphate, magnesium sulphate, magnesium hydrogen sulphate, aluminium sulphate and aluminium hydrogen sulphate. In one embodiment of the present disclosure, the anionic salts are chosen from ammonium sulphate and sodium sulphate.

The concentration of the at least one salt should, for example, be sufficient to induce the precipitation of the at least one high molecular weight water-soluble drawing polymer formed in the polymerization medium, and may be up to the saturation concentration of each salt. To obtain such a precipitation, the at least one salt can be, for instance, present in an amount equal to or greater than 10% by weight, such as equal to or greater than 15% by weight, and for example, less than 50% by weight, relative to the total weight of the polymer solution or dispersion. The saline aqueous solution may also comprise monovalent salts such as sodium chloride and ammonium chloride.

The heterogeneous free-radical polymerization in aqueous medium as described above may be accompanied by a large increase in the viscosity of the reaction medium, which can be reflected by at least one indicator, such as difficulties in stirring, a lack of homogeneity of the reaction medium and an increase in the particle size of the polymer particles formed. To prevent such an increase in viscosity, it has been proposed, in European Patent Application No. EP 0 943 628, to add to the polymerization medium at least one agent for preventing the increase in viscosity of the reaction medium during polymerization.

The at least one high molecular weight water-soluble drawing polymer used in the present disclosure can be prepared, for example, in the presence of at least one agent for preventing the increase in viscosity. Among the agents for preventing the increase in viscosity of the reaction medium, non-limiting mention may be made of, for example:

(1) polycarboxylic acids and salts thereof,

(2) polyphenols,

(3) cyclic compounds comprising a hydroxyl group and a carboxyl group, and salts thereof,

(4) gluconic acid and salts thereof,

(5) the reaction products obtained by reacting a methoxyhydroquinone and/or a cationic (meth)acrylic monomer with a free-radical-generating compound, under an oxidizing atmosphere,

(6) the reaction products obtained by reacting a cationic (meth)acrylic polymer with a free-radical-generating compound, under an oxidizing atmosphere,

(7) the reaction products obtained by reacting a cationic (meth)acrylic polymer with an oxidizing agent,

and mixtures thereof.

The addition of at least one agent for preventing the increase in viscosity as described above can make it possible to perform the polymerization of the water-soluble monomers described above with a low-power stirrer while at the same time avoiding the formation of coarse particles. The agents for preventing an increase in viscosity can be, for example, soluble in the aqueous reaction medium.

Non-limiting examples of (1) polycarboxylic acids and salts thereof that may be mentioned include oxalic acid, adipic acid, tartaric acid, malic acid and phthalic acid, and the salts thereof.

Non-limiting examples of (2) polyphenols that may be mentioned, for instance, include resorcinol and pyrogallol.

Non-limiting examples of (3) cyclic compounds comprising a hydroxyl group and a carboxyl group, and salts thereof that may be mentioned include m-hydroxybenzoic acid, p-hydroxybenzoic acid, salicylic acid, gallic acid and tannic acid, and the salts of these acids.

Non-limiting examples of (4) gluconic acid and salts thereof that may be mentioned include sodium gluconate, potassium gluconate, ammonium gluconate and various amine salts of gluconic acid.

Non-limiting examples of (5) the reaction products obtained by reacting a methoxyhydroquinone and/or a cationic (meth)acrylic monomer with a free-radical-generating compound, under an oxidizing atmosphere that may be mentioned include those obtained by reacting a free-radical-generating compound, under a stream of oxygenated gas, in a solution comprising methoxyhydroquinone and/or a cationic (meth)acrylic monomer. The free-radical-generating compound may be an initiator commonly used for free-radical polymerization. Further non-limiting examples that may be mentioned include water-soluble azo initiators such as 2,2′-azobis(2-amidinopropane) hydrochloride sold, for example, under the name V-50 by the company Wako Chemical Industries, or 2,2′-azobis[2-(2-imidazolin-2-yl)propane] hydrochloride sold, for example, under the trade name VA-044 by the company Wako Chemical Industries, or an initiator from the group of water-soluble redox agents, such as the ammonium persulphate/sodium hydrogen sulphite combination.

An agent for preventing an increase in viscosity (6) obtained by reacting a cationic (meth)acrylic polymer with a free-radical-generating compound, under an oxidizing atmosphere, may be obtained by reacting a free-radical initiator, under a oxygenated atmosphere, with a dispersant according to the present disclosure. The polymerization initiator may be a water-soluble azo initiator or a water-soluble redox agent as described above.

The reaction products (7) obtained by reacting a cationic (meth)acrylic polymer with an oxidizing agent may be obtained in the form of oxidized polymers of low molecular mass by oxidation of a cationic dispersant according to the present disclosure obtained by polymerization of a cationic (meth)acrylic monomer, using hydrogen peroxide or a halogen as oxidizing agent.

Among cationic (meth)acrylic monomers used for the preparation of agents for preventing an increase in viscosity of categories (5), (6), and (7) as described above, non-limiting examples that may be mentioned include dimethylaminoethyl (meth)acrylate hydrochloride or sulphate, (meth)acryloyloxyethyl-trimethylammonium chloride, (meth)acryloyloxyethyl-dimethylbenzylammonium chloride, the hydrochloride or sulphate derived from N-dimethylaminopropyl(meth)-acrylamide, (meth)acrylamidopropyltrimethylammonium chloride, dimethylaminohydroxypropyl (meth)acrylate chloride or sulphate, (meth )acryloyloxyhydroxypropyl-trimethylammonium chloride and (meth)acryloyloxy-hydroxypropyldimethylbenzylammonium chloride.

The at least one agent for preventing an increase in viscosity (1) to (7) as disclosed herein may be present in an amount ranging, for example, from 10 ppm to 10,000 ppm, relative to the total weight of the reaction solution.

The water-soluble monomers polymerized by heterogeneous free-radical polymerization to obtain the at least one high molecular weight water-soluble drawing polymer are monomers comprising at least one ethylenic double bond, for example a vinyl, acrylic or allylic double bond. The water-soluble monomers may be cationic, anionic or nonionic and may be used as a mixture, as long as at least one monomer is cationic.

Non-limiting examples of water-soluble anionic monomers that may be mentioned include acrylic acid, methacrylic acid, acrylamido-2-methylpropanesulphonic acid and itaconic acid. These anionic monomers are at least partially neutralized in the form of a salt of an alkali metal (for example sodium or potassium), of an alkaline-earth metal, of ammonium or of an organic amine such as an alkanolamine, for instance, ethanolamine.

Non-limiting examples of water-soluble nonionic monomers that may be mentioned include acrylamide, methacrylamide, N-vinylformamide, N-vinylacetonamide, hydroxypropyl acrylate and hydroxypropyl methacrylate.

The water-soluble cationic monomers can be, for example, chosen from di(C₁₋₄ alkyl)diallylammonium salts and the compounds of formula (I) 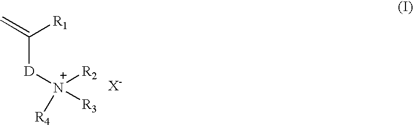
wherein

R₁ is chosen from a hydrogen atom and methyl groups,

R₂ and R₃, which may be identical or different, are chosen from hydrogen atoms and linear and branched C₁₋₄ alkyl groups,

R₄ is chosen from a hydrogen atom, linear and branched C₁₋₄ alkyl groups, and aryl groups,

D is chosen from units of the following formula: 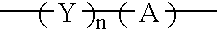
wherein Y is chosen from amide (—CO—NH—), ester (—O—CO— or —CO—O—), urethane (—O—CO—NH—) and urea (—NH—CO—NH—) functional groups,

A is chosen from linear, branched and cyclic C₁₋₁₀ alkylene groups, which may be substituted or interrupted with a divalent aromatic or heteroaromatic ring, or which may be interrupted with a hetero atom chosen from O, N, S and P, and which may comprise a functional group chosen from ketone, amide, ester, urethane and urea functional groups,

n is 0 or 1, and

X⁻is an anionic counterion such as a chloride or sulphate ion.

Non-limiting examples of water-soluble cationic monomers that may be mentioned include dimethylaminoethyl (meth)acrylate hydrochloride or sulphate, (meth )acryloyloxyethyltrimethylammonium chloride, (meth )acryloyloxyethyldimethylbenzylammonium chloride, N-dimethylaminopropyl(meth)acrylamide hydrochloride or sulphate, (meth)acrylamidopropyltrimethylammonium chloride, (meth)acrylamidopropyldimethylbenzylammonium chloride, dimethylaminohydroxypropyl (meth)acrylate hydrochloride or sulphate, (meth)acryloyloxyhydroxy-propyltrimethylammonium chloride, (meth)acryloyloxy-hydroxypropyldimethylbenzylammonium chloride and dimethyldiallylammonium chloride.

In one embodiment of the present disclosure, for example, the at least one high molecular weight water-soluble drawing polymer is obtained by heterogeneous free-radical polymerization of a monomer mixture comprising from 0 mol % to 30 mol % of acrylic acid, from 0 mol % to 95.5 mol % of acrylamide, and from 0.5 mol % to 100 mol % of at least one cationic monomer of formula (I).

In another embodiment of the present disclosure, the at least one high molecular weight water soluble drawing polymer is obtained by heterogeneous free-radical polymerization of a mixture of monomers comprising from 0 mol % to 95.5 mol % of acrylamide and from 4.5 mol % to 100 mol % of at least one cationic monomer of formula (I).

According to still another embodiment, of the present disclosure, the at least one high molecular weight water-soluble drawing polymer is obtained by polymerization of a monomer mixture comprising acrylic acid and a cationic monomer of formula (I), wherein the number of moles of cationic monomer of formula (I) is greater than the number of moles of acrylic acid.

Among the water-soluble polyelectrolytes that may be used as disclosed herein, non-limiting mention may be made, for example, of those polymerized using monomer mixtures comprising:

1) 10 mol % of acryloyloxyethyldimethylbenzyl-ammonium chloride and 90 mol % of acrylamide;

2) 30 mol % of acryloyloxytrimethylammonium chloride, 50 mol % of acryloyloxyethyldimethylbenzyl-ammonium chloride and 20 mol % of acrylamide;

3) 10 mol % of acryloyloxyethyltrimethylammonium chloride and 90 mol % of acrylamide;

4) 30 mol % of diallyldimethylammonium chloride and 70 mol % of acrylamide.

As noted above, the at least one water-soluble drawing polymer used in the present disclosure has a high molecular weight. In the context of this disclosure, the term “high molecular weight” refers to a weight-average molecular mass of greater than or equal to 1,000,000, for example ranging from 1,000,000 to 50,000,000. This weight-average molecular mass is determined via the RSV (Reduced Specific Viscosity) method as defined in Principles of Polymer Chemistry, Cornell University Press, Ithaca, N.Y., 1953, chapter VII entitled “Determination of Molecular Weight,” pages 266-316.

The concentration of the high molecular weight water-soluble drawing polymer dispersion or solution can be chosen such that, for instance, the at least one high molecular water-soluble drawing polymer is present in an amount ranging from 0.01 % to 10% by weight, such as from 0.05% to 5% by weight, relative to the total weight of the final composition.

The at least one water-soluble drawing polymer whose particles are present as a dispersion in a saline aqueous solution in (a1) can be present in an amount ranging from 0.01% to 20% by weight, relative to the total weight of the dispersion.

The at least one water-soluble drawing polymer present as a saline aqueous solution in (a2) can be present in the composition in an amount ranging from 0.01% to 20% by weight, relative to the total weight of the solution.

The at least one drawing polymer can be present, for example, in an amount ranging from 0.01% to 10% by weight, such as from 0.05% to 5% by weight, relative to the total weight of the final composition.

The at least one detergent surfactant is chosen from anionic, amphoteric, nonionic and zwitterionic surfactants.

Thus, according to the present disclosure, the at least one detergent surfactant can be present in a total amount ranging from 4% to 50% by weight, such as from 6% to 30% by weight, and for instance, from 8% to 25% by weight, relative to the total weight of the final composition.

Among the surfactants that can be used according to the present disclosure, non-limiting mention may be made of the following:

(i) Anionic Surfactant(s):

In the context of the present disclosure, the nature of the anionic surfactants is not a critical feature. Thus, as non-limiting examples of anionic surfactants that may be used, alone or as mixtures, in the context of the present disclosure, mention may be made of salts (such as alkaline salts, for instance sodium salts, ammonium salts, amine salts, amino alcohol salts or magnesium salts) of the following compounds: alkyl sulphates, alkyl ether sulphates, alkylamido ether sulphates, alkylaryl polyether sulphates, monoglyceride sulphates, alkyl sulphonates, alkyl phosphates, alkylamide sulphonates, alkylaryl sulphonates, α-olefin sulphonates, paraffin sulphonates, alkyl sulphosuccinates, alkyl ether sulphosuccinates, alkylamide sulphosuccinates, alkyl sulphosuccinamates, alkyl sulphoacetates, alkyl ether phosphates, acyl sarcosinates, acyl isethionates and N-acyltaurates, the alkyl or acyl radical of all of these various compounds comprising, for example, from 12 to 20 carbon atoms, and the aryl radical may be, for example, a phenyl or benzyl group. Among the anionic surfactants that may also be used, further non-limiting mention may also be made of fatty acid salts such as the salts of oleic, ricinoleic, palmitic and stearic acids, coconut oil acid or hydrogenated coconut oil acid, and acyl lactylates in which the acyl radical comprises from 8 to 20 carbon atoms. Use may also be made of weakly anionic surfactants, such as alkyl-D-galactosiduronic acids and their salts, and also polyoxyalkylenated carboxylic ether acids and their salts, for instance, those comprising from 2 to 50 ethylene oxide groups, and mixtures thereof. Anionic surfactants of the polyoxyalkylenated carboxylic ether acid or salt type can be, for example, chosen from those of formula (1):
R₁—(OC₂H₄)ₙ—OCH₂COOA (1)
wherein:

R₁ is chosen from alkyl, alkylamido and alkaryl groups wherein the alkyl radical comprises from 6 to 20 carbon atoms, and for example, the aryl may be a phenyl;

n is an integer or decimal number (average value) ranging from 2 to 24, such as from 3 to 10, and

A is chosen from a hydrogen atom, ammonium ions, Na ions, K ions, Li ions, Mg ions, and monoethanolamine and triethanolamine groups.

Mixtures of compounds of formula (1) can also be used, for instance, mixtures in which the groups R₁ are different.

Compounds of formula (1) are sold, for example, by the company Chem Y under the name Akypo (NP40, NP70, OP40, OP80, RLM25, RLM38, RLMQ 38 NV, RLM 45, RLM 45 NV, RLM 100, RLM 100 NV, RO 20, RO 90, RCS 60, RS 60, RS 100, RO 50) or by the company Sandoz under the name Sandopan (DTC Acid, DTC).

(ii) Nonionic Surfactant(s):

Nonionic surfactants are likewise compounds that are well known per se (see, for example in this respect, Handbook of Surfactants by M. R. Porter, published by Blackie & Son (Glasgow and London), 1991, pp. 116-178) and, in the context of the present. disclosure, their nature is not a critical feature. Thus, among the nonionic surfactants that may be used, non-limiting mention may be made of polyethoxylated, polypropoxylated or polyglycerolated fatty alcohols, polyethoxylated, polypropoxylated or polyglycerolated fatty a-diols, polyethoxylated, polypropoxylated or polyglycerolated fatty alkylphenols and polyethoxylated, polypropoxylated or polyglycerolated fatty acids, all having a fatty chain comprising, for example, 8 to 18 carbon atoms, it being possible for the number of ethylene oxide or propylene oxide groups to range, for instance, from 2 to 50 and for the number of glycerol groups to range, for example, from 2 to 30. Non-limiting mention may also be made of copolymers of ethylene oxide and of propylene oxide, condensates of ethylene oxide and of propylene oxide with fatty alcohols; polyethoxylated fatty amides comprising, for example, from 2 to 30 mol of ethylene oxide, polyglycerolated fatty amides comprising on average 1 to 5, such as 1.5 to 4, glycerol groups; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. In one embodiment of the present disclosure, the nonionic surfactants are chosen from alkylpolyglycosides.

(iii) Amphoteric and Zwitterionic Surfactant(s):

The nature of the amphoteric and/or zwitterionic surfactants is not a critical feature in the context of the present disclosure. Non-limiting examples of such surfactants that can be used include, for instance aliphatic secondary or tertiary amine derivatives in which the aliphatic radical is chosen from linear and branched chains comprising from 8 to 18 carbon atoms and comprising at least one water-soluble anionic group (for example carboxylate, sulphonate, sulphate, phosphate or phosphonate); non-limiting mention may also be made of (C₈-C₂₀)alkylbetaines, sulphobetaines, (C₈-C₂₀)alkylamido(C₁-C₆)-alkylbetaines or (C₈-C₂₀)alkylamido(C₁-C₆)alkylsulphobetaines.

Among the amine derivatives, non-limiting mention may be made of the products sold under the name Miranol, as described in U.S. Pat. Nos. 2,528,378 and 2,781,354 and classified in the CTFA dictionary, 3rd edition, 1982, under the names Amphocarboxyglycinates and Amphocarboxypropionates, with the respective structures (2) and (3):
R₂—CONHCH₂CH₂—N⁺(R₃)(R₄)(CH₂COO⁻) (2)
wherein:
R₂ is chosen from alkyl radicals of an acid R₂—COOH present in hydrolysed coconut oil, and heptyl, nonyl and undecyl radicals, R₃ is a β-hydroxyethyl group and R₄ is a carboxymethyl group; and
   R₂′—CONHCH₂CH₂—N(B)(C) (3)
   wherein:

B is —CH₂CH₂OX′, C is —(CH₂)_{z}—Y′, z is equal to 1 or 2,

X′ is chosen from —CH₂CH₂—COOH groups and a hydrogen atom,

Y′ is chosen from —COOH and —CH₂—CHOH—SO₃H radicals,

R₂′ is chosen from alkyl radicals of an acid R₂′—COOH present in coconut oil or in hydrolysed linseed oil; alkyl radicals, such as C₇, C₉, C₁₁ and C₁₃ alkyl radicals; C₁₇ alkyl radicals and its iso form, and unsaturated C₁₇ radicals.

For example, non-limiting mention may be made of the cocoamphocarboxyglycinate sold under the trade name Miranol C2M concentrate by the company Miranol.

Mixtures of surfactants, for instance, mixtures of anionic surfactants, mixtures of anionic surfactants and of amphoteric, cationic or nonionic surfactants, or mixtures of cationic surfactants with nonionic or amphoteric surfactants, may be used in the compositions in accordance with the present disclosure. In one embodiment of the present disclosure, the surfactants are present in a mixture comprising at least one anionic surfactant and at least one amphoteric surfactant.

The at least one anionic surfactant, when present, can be present, for example, in a total amount ranging from 3% to 40% by weight, relative to the total weight of the cosmetic composition, for instance, from 5% to 35% by weight, such as from 8% to 25% by weight.

The at least one amphoteric and/or nonionic surfactant, when present, can be present in a total amount, for example, ranging from 0.5% to 20% by weight, such as from 1% to 15% by weight, relative to the total weight of the composition.

Cationic Polymers

The cationic polymers used herein, are, as an initial matter, different from the drawing polymer discussed above, and may be chosen from all those already known per se as improving the cosmetic properties of the hair, e.g., those described in European Patent Application No. EP-A-0 337 354 and in French Patent Application Nos. FR-A-2,270,846, 2,383,660, 2,598,611, 2,470,596 and 2,519,863.

Also as used herein, the term “cationic polymer” is understood to mean any polymer comprising cationic groups and/or groups that may be ionized into cationic groups.

Among the cationic polymers that may be used, non-limiting mention may be made of those comprising units comprising primary, secondary, tertiary and/or quaternary amine groups that either may form part of the main polymer chain or may be borne by a side substituent directly attached thereto.

The cationic polymers can have, for example, a number-average or weight-average molar mass ranging from 500 to 5×10⁶, such as from 10³ to 3×10⁶.

Further among the cationic polymers that may be used, non-limiting mention may also be made, for instance, of polymers of the polyamine, polyamino amide and polyquaternary ammonium type. These are known products.

Still further among the polymers of the polyamine, polyamino amide and polyquaternary ammonium type that may be used in accordance with the present disclosure, non-limiting mention may be made, of those described in French Patent Nos. 2,505,348 and 2,542,997. Among these polymers, non-limiting examples include:

(1) homopolymers or copolymers derived from acrylic or methacrylic esters or amides and comprising at least one unit of the formulae: 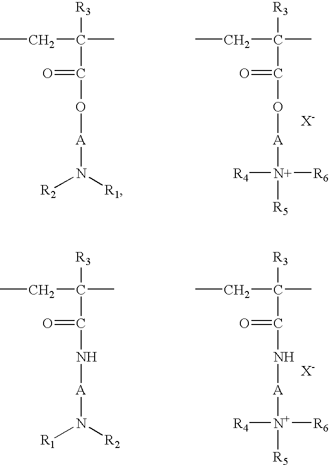
wherein:

R₃, which may be identical or different, is chosen from hydrogen atoms and CH₃ radicals;

A, which may be identical or different, is chosen from linear and branched alkyl groups of 1 to 6 carbon atoms, such as 2 to 3 carbon atoms, and hydroxyalkyl groups of 1 to 4 carbon atoms;

R₄, R₅ and R₆, which may be identical or different, are chosen from alkyl groups comprising from 1 to 18 carbon atoms, such as alkyl groups comprising from 1 to 6 carbon atoms, and benzyl radicals;

R₁ and R₂, which may be identical or different, are chosen from hydrogen atoms and alkyl groups comprising from 1 to 6 carbon atoms, for instance methyl or ethyl groups;

X⁻ is an anion derived from a mineral or organic acid, such as a methosulphate anion or a halide such as chloride or bromide.

The copolymers of family (1) can also comprise at least one unit derived from comonomers that are chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of family (1), non-limiting mention may be made of:
copolymers of acrylamide and of dimethylaminoethyl methacrylate quaternized with dimethyl sulphate or with a dimethyl halide, such as the product sold under the name Hercofloc by the company Hercules,
copolymers of acrylamide and of methacryloyloxyethyltrimethylammonium chloride described, for example, in Patent Application No. EP-A-080 976 and sold under the name Bina Quat P 100 by the company Ciba Geigy,
copolymers of acrylamide and of methacryloyloxyethyltrimethylammonium methosulphate sold under the name Reten by the company Hercules,
quaternized or non-quaternized vinylpyrrolidone/dialkylaminoalkyl acrylate or methacrylate copolymers. These polymers are described in detail in French Patent Nos. 2,077,143 and 2,393,573,
dimethylaminoethyl methacrylate/vinylcaprolactam/vinylpyrrolidone terpolymers,
vinylpyrrolidone/methacrylamidopropyldimethylamine copolymers,
and quaternized vinylpyrrolidone/dimethylaminopropylmethacrylamide copolymers.

(2) cationic polysaccharides, for instance, cationic celluloses and cationic galactomannan gums. Among the cationic polysaccharides that may be used, non-limiting mention may be made of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are described in French Patent No. 1,492,597. These polymers are also defined in the CTFA dictionary as hydroxyethylcellulose quaternary ammoniums that have reacted with an epoxide substituted with a trimethylammonium group.

The cationic cellulose copolymers or cellulose derivatives grafted with a water-soluble quaternary ammonium monomer are described for example, in U.S. Pat. No. 4,131,576, such as hydroxyalkylcelluloses, for instance hydroxymethyl-, hydroxyethyl- or hydroxypropylcelluloses grafted for instance, with a methacryloylethyltrimethylammonium, methacrylamidopropyltrimethylammonium or dimethyldiallylammonium salt.

The cationic galactomannan gums are described, for example, in U.S. Pat. Nos. 3,589,578 and 4,031,307, for instance, guar gums comprising trialkylammonium cationic groups. Use can be made, for example, of guar gums modified with a salt (e.g. chloride) of 2,3-epoxypropyltrimethylammonium.

(3) polymers comprising of piperazinyl units and of divalent alkylene or hydroxyalkylene radicals comprising straight or branched chains, optionally interrupted by oxygen, sulphur or nitrogen atoms or by aromatic or heterocyclic rings, and also the oxidation and/or quaternization products of these polymers. Such polymers are described, for example, in French Patent Nos. 2,162,025 and 2,280,361.

(4) water-soluble polyamino amides prepared, for instance, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound that is reactive with respect to a bis-halohydrin, a bis-azetidinium, a bis-haloacyldiamine, a bis-alkyl halide, an epihalohydrin, a diepoxide or a bis-unsaturated derivative; the crosslinking agent being used in an amount ranging from 0.025 mol to 0.35 mol per amine group of the polyamino amide; these polyamino amides can be alkylated or, if they comprise at least one tertiary amine functional group, they can be quaternized. Such polymers are described, for example, in French Patent Nos. 2,252,840 and 2,368,508.

(5) polyamino amide derivatives resulting from the condensation of polyalkylene polyamines with polycarboxylic acids followed by alkylation with difunctional agents. Non-limiting mention may be made, for example, of adipic acid/dialkylamino-hydroxyalkyldialkylenetriamine polymers in which the alkyl radical comprises from 1 to 4 carbon atoms, such as methyl, ethyl or propyl. Such polymers are described for instance, in French Patent No. 1,583,363.

Among these derivatives, non-limiting mention may be made, for example, of the adipic acid/dimethylaminohydroxypropyl/diethylenetriamine polymers sold under the name “Cartaretine F, F4 or F8” by the company Sandoz.

(6) polymers obtained by reaction of a polyalkylene polyamine comprising two primary amine groups and at least one secondary amine group with a dicarboxylic acid chosen from diglycolic acid and saturated aliphatic dicarboxylic acids comprising from 3 to 8 carbon atoms. The molar ratio between the polyalkylene polyamine and the dicarboxylic acid can range from 0.8:1 to 1.4:1; the polyamino amide resulting therefrom being reacted with epichlorohydrin in a molar ratio of epichlorohydrin relative to the secondary amine group of the polyamino amide ranging from 0.5:1 to 1.8:1. Such polymers are described, for instance, in U.S. Pat. Nos. 3,227,615 and 2,961,347.

Polymers of this type are sold, for example, under the name “Hercosett 57” by the company Hercules Inc., by the company Hercules in the case of the adipic acid/epoxypropyl/diethylenetriamine copolymer.

(7) cyclopolymers of alkyldiallylamine or of dialkyldiallylammonium, such as the homopolymers or copolymers comprising, as the main constituent of the chain, units of formulae (I) or (I′): 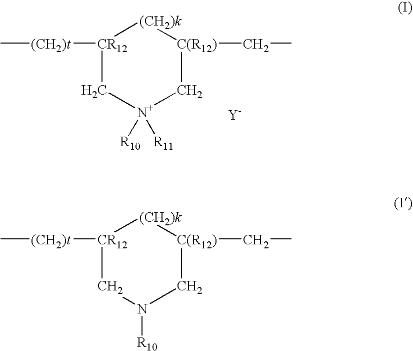
wherein k and t are equal to 0 or 1, the sum k+t being equal to 1; R₁₂ is chosen from a hydrogen atom and methyl radicals; R₁₀ and R₁₁, which may be identical or different, are chosen from alkyl groups comprising from 1 to 6 carbon atoms, hydroxyalkyl groups in which the alkyl group may comprise, for example, 1 to 5 carbon atoms, and lower (C₁-C₄) amidoalkyl groups, or R₁₀ and R₁₁ can form, together with the nitrogen atom to which they are attached, heterocyclic groups such as piperidyl or morpholinyl; Y⁻ is an anion such as bromide, chloride, acetate, borate, citrate, tartrate, bisulphate, bisulphite, sulphate or phosphate. These polymers are described, for example, in French Patent No. 2,080,759 and in its Certificate of Addition No. 2,190,406.

In one embodiment of the present disclosure, R₁₀ and R₁₁, which may be identical or different, are chosen from alkyl groups comprising from 1 to 4 carbon atoms.

Among the polymers defined above, non-limiting mention may be made for example, of the dimethyldiallylammonium chloride homopolymer sold under the name “Merquat 100” by the company Nalco (and its homologues of low weight-average molar mass) and copolymers of diallyldimethylammonium chloride and of acrylamide.

(8) diquaternary ammonium polymers comprising repeating units of formula (II): 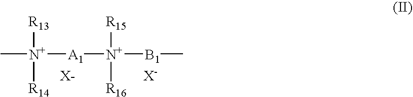
wherein:

R₁₃, R₁₄, R₁₅ and R₁₆, which may be identical or different, are chosen from aliphatic, alicyclic and arylaliphatic radicals comprising from 1 to 20 carbon atoms, and lower hydroxyalkylaliphatic radicals, or alternatively R₁₃, R₁₄, R₁₅ and R₁₆, together or separately, form, with the nitrogen atoms to which they are attached, heterocycles optionally comprising a second hetero atom other than nitrogen, or alternatively R₁₃, R₁₄, R₁₅ and R₁₆ are chosen from linear and branched C₁-C₆ alkyl radicals substituted with a nitrile, ester, acyl or amide group or a group —CO—O—R₁₇-D or —CO—NH—R₁₇-D where R₁₇ is an alkylene and D is a quaternary ammonium group;

A₁ and B₁ are chosen from linear and branched, saturated and unsaturated polymethylene groups comprising from 2 to 20 carbon atoms, which may comprise, linked to or intercalated in the main chain, at least one aromatic ring or at least one entity chosen from oxygen atoms, sulphur atoms, and sulphoxide, sulphone, disulphide, amino, alkylamino, hydroxyl, quaternary ammonium, ureido, amide and ester groups, and

X⁻is an anion derived from a mineral or organic acid;

A₁, R₁₃ and R₁₅ can form, with the two nitrogen atoms to which they are attached, a piperazine ring; in addition, if A₁ is chosen from linear and branched, saturated and unsaturated alkylene and hydroxyalkylene radicals, then B₁ can also be chosen from (CH₂)ₙ—CO-D-OC—(CH₂)ₙ— groups

in which D is chosen from:

a) glycol groups of formula: —O-Z-O—, where Z is chosen from linear and branched hydrocarbon-based radicals or groups of one of the following formulae:
—(CH₂—CH₂—O)ₓ—CH₂—CH₂—
—[CH₂—CH(CH₃)—O]_{y}—CH₂—CH(CH₃)—
where x and y are integers ranging from 1 to 4, representing a defined and unique degree of polymerization, or any number from 1 to 4 representing an average degree of polymerization;

b) bis-secondary diamine groups, such as a piperazine derivative;

c) bis-primary diamine groups of formula: —NH—Y—NH—, where Y is chosen from linear and branched hydrocarbon-based radicals, or alternatively the divalent radical
—CH₂—CH₂—S—S—CH₂—CH₂—; and

d) ureylene groups of formula: —NH—CO—NH—;

For example, X⁻ may be anion such as chloride or bromide.

These polymers can have a number-average molar mass ranging from 1,000 to 100,000.

Polymers of this type are described, for instance, in French Patent Nos. 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is also possible, for example, to use polymers that comprising repeating units of formula (a): 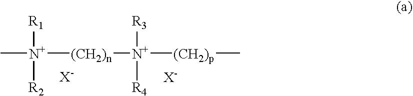
wherein R₁, R₂, R₃ and R₄, which may be identical or different, are chosen from alkyl and hydroxyalkyl radicals comprising from 1 to 4 carbon atoms, n and p are integers ranging from 2 to 20, and X⁻ is an anion derived from a mineral or organic acid.

One compound of formula (a), for example, may be where R₁, R₂, R₃ and R₄ are methyl radicals, n is equal to 3, p is equal to 6 and X is a Cl atom, which is known as Hexadimethrine chloride according to the INCI (CTFA) nomenclature.

(9) polyquaternary ammonium polymers comprising of units of formula (III): 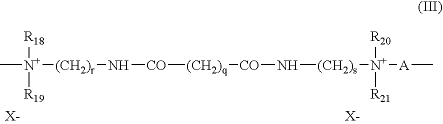
wherein:

R₁₈, R₁₉, R₂₀ and R₂₁, which may be identical or different, are chosen from hydrogen atoms and methyl, ethyl, propyl, β-hydroxyethyl, β-hydroxypropyl and —CH₂CH₂(OCH₂CH₂)ₚOH radicals,

p is an integer ranging from 0 to 6, with the proviso that R₁₈, R₁₉, R₂₀ and R₂₁ are not simultaneously all hydrogen atoms,

r and s, which may be identical or different, are integers ranging from 1 to 6,

q is an integer ranging from 0 to 34,

X⁻ is an anion such as a halide,

A is chosen from dihalide radicals and —CH₂—CH₂—O—CH₂—CH₂— radicals.

Such compounds are described, for example, in Europen Patent Application EP-A-122 324. Among these products, non-limiting mention may be made, for example, of the products “Mirapol® A 15”, “Mirapole® AD1”, “Mirapolo® AZ1” and “Mirapol® 175” sold by the company Miranol.

(10) quaternary polymers of vinylpyrrolidone and of vinylimidazole, for instance the products sold under the names Luviquat® FC 905, FC 550 and FC 370 by the company BASF.

(11) crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, such as methylenebisacrylamide.

Other cationic polymers that may be used in the context of the present disclosure include cationic proteins or cationic protein hydrolyzates, polyalkyleneimines, such as polyethyleneimines, polymers comprising vinylpyridine or vinylpyridinium units, condensates of polyamines and of epichlorohydrin, polyquaternary ureylenes and chitin derivatives, for example chitosans or salts thereof; the salts that may be used include, for instance chitosan acetate, lactate, glutamate, gluconate or pyrrolidonecarboxylate. Among these compounds, non-limiting mention may be made of chitosan with a degree of deacetylation of 90% by weight, and the chitosan pyrrolidonecarboxylate sold under the name Kytamer® PC by the company Amerchol.

Among all the cationic polymers that may be used in the context of the present disclosure, further non-limiting examples may be made of cationic cyclopolymers, such as the dimethyldiallylammonium chloride homopolymers or copolymers sold under the names “Merquat 100”, “Merquat 550” and “Merquat S” by the company Nalco, quaternary polymers of vinylpyrrolidone and of vinylimidazole, crosslinked homopolymers or copolymers of methacryloyloxy(C₁-C₄)alkyltri(C₁-C₄)alkylammonium salts, and the chitosan pyrrolidonecarboxylate sold under the name Kytamer® PC by the company Amerchol, and mixtures thereof.

Amphoteric Polymers

The amphoteric polymers that may be used as disclosed herein can be chosen from polymers comprising units K and M randomly distributed in the polymer chain, in which K is chosen from units derived from a monomer comprising at least one basic nitrogen atom and M is chosen from units derived from an acidic monomer comprising at least one group chosen from carboxylic and sulphonic groups, or alternatively K and M can be chosen from groups derived from zwitterionic carboxybetaine and sulphobetaine monomers. K and M may also be chosen from cationic polymer chains comprising primary, secondary, tertiary or quaternary amine groups, wherein at least one of the amine groups bears a carboxylic or sulphonic group linked via a hydrocarbon-based radical, or alternatively K and M form part of a chain of a polymer comprising an α,β-dicarboxylic ethylene unit in which one of the carboxylic groups has been made to react with a polyamine comprising at least one amine group chosen from primary and secondary amine group.

Among the amphoteric polymers corresponding to the above definition, non-limiting mention may be made, for example, of those chosen from the following polymers:

(1) polymers resulting from the copolymerization of a monomer derived from a vinyl compound bearing a carboxylic group such as, for instance, acrylic acid, methacrylic acid, maleic acid, α-chloroacrylic acid, and a basic monomer derived from a substituted vinyl compound comprising at least one basic atom, such as, for example, dialkylaminoalkyl methacrylate and acrylate, dialkylaminoalkylmethacrylamide and -acrylamide. Such compounds are described in U.S. Pat. No. 3,836,537. Non-limiting mention may also be made of the sodium acrylate/acrylamidopropyltrimethylammonium chloride copolymer sold under the name Polyquart KE 3033 by the company Cognis. The vinyl compound may also be a dialkyldiallylammonium salt such as dimethyldiallylammonium salt (for example chloride). The copolymers of acrylic acid and of the latter monomer are sold under the names Merquat 280 and Merquat 295 by the company Nalco.

(2) Polymers comprising units derived from:

a) at least one monomer chosen from acrylamides and methacrylamides substituted on the nitrogen with an alkyl radical,

b) at least one acidic comonomer comprising at least one reactive carboxylic group, and

c) at least one basic comonomer such as esters comprising primary, secondary, tertiary and quaternary amine substituents of acrylic and methacrylic acids and the product of quaternization of dimethylaminoethyl methacrylate with dimethyl or diethyl sulphate.

For example, the N-substituted acrylamides or methacrylamides as disclosed herein may be groups in which the alkyl radicals comprise from 2 to 12 carbon atoms, such as N-ethylacrylamide, N-tert-butylacrylamide, N-tert-octylacrylamide, N-octylacrylamide, N-decylacrylamide, N-dodecylacrylamide and the corresponding methacrylamides. The acidic comonomers may be chosen, for example, from acrylic acid, methacrylic acid, crotonic acid, itaconic acid, maleic acid and fumaric acid and alkyl monoesters, comprising from 1 to 4 carbon atoms, of maleic or fumaric acids or anhydrides. For further example, the basic comonomers may be chosen from aminoethyl, butylaminoethyl, N,N′-dimethylaminoethyl and N-tert-butylaminoethyl methacrylates. The copolymers whose CTFA (4th edition, 1991) name is Octylacrylamide/acrylates/butylaminoethyl methacrylate copolymer may also be used.

(3) Polyamino amides that are crosslinked and alkylated partially or totally derived from polyamino amides of general formula: 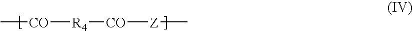
wherein R₄ is chosen from divalent radicals derived from a saturated dicarboxylic acid, mono- or dicarboxylic aliphatic acids comprising an ethylenic double bond, esters of lower alkanols comprising from 1 to 6 carbon atoms of these acids and radicals derived from the addition of any one of the acids to a bis(primary) or bis(secondary) amine, and Z is chosen from bis(primary), mono- and bis(secondary) polyalkylene-polyamine radicals, and may, for example be chosen from:

a) in an amount ranging from 60 mol % to 100 mol %, the radical

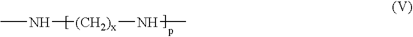

wherein x is equal to 2 and p is equal to 2 or 3, or alternatively x is equal to 3 and p is equal to 2

this radical being derived from diethylenetriamine, from triethylenetetraamine or from dipropylenetriamine;
b) in an amount ranging from 0 mol % to 40 mol %, the radical (V) above in which x is equal to 2 and p is equal to 1 and which is derived from ethylenediamine, or the radical derived from piperazine: 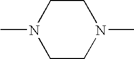
c) in an amount ranging from 0 mol % to 20 mol %, the —NH—(CH₂)₆—NH— radical derived from hexamethylenediamine, these polyamino amines being crosslinked by addition of a difunctional crosslinking agent chosen from epihalohydrins, diepoxides, dianhydrides and bis-unsaturated derivatives, using from 0.025 mol to 0.35 mol of crosslinking agent per amine group of the polyamino amide and alkylated by the action of acrylic acid, chloroacetic acid or an alkane sultone, or salts thereof.

The saturated carboxylic acids may be chosen, for example, from acids comprising from 6 to 10 carbon atoms, such as adipic acid, 2,2,4-trimethyladipic acid and 2,4,4-trimethyladipic acid, terephthalic acid and acids comprising an ethylenic double bond such as, for example, acrylic acid, methacrylic acid and itaconic acid. The alkane sultones used in the alkylation may be, for example, propane sultone or butane sultone, and the salts of the alkylating agents may be, for instance, the sodium or potassium salts.

(4) Polymers comprising zwitterionic units of formula (VI): 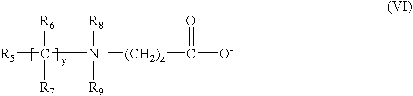
wherein R₅ is chosen from polymerizable unsaturated groups such as acrylate, methacrylate, acrylamide and methacrylamide groups, y and z are integers from 1 to 3, R₆ and R₇ are chosen from hydrogen atoms, and methyl, ethyl and propyl groups, R₈ and R₉ are chosen from hydrogen atoms and alkyl radicals such that the sum of the carbon atoms in R₈ and R₉ does not exceed 10.

The polymers comprising such units may also comprise units derived from non-zwitterionic monomers such as dimethyl or diethylaminoethyl acrylate or methacrylate, or alkyl acrylates or methacrylates, acrylamides or methacrylamides, or vinyl acetate.

By way of non-limiting example, mention may be made of the copolymer of butyl methacrylate/dimethylcarboxymethylammonioethyl methacrylate.

(5) Polymers derived from chitosan comprising monomer units chosen from those of formulae (VII), (VIII) and (IX): 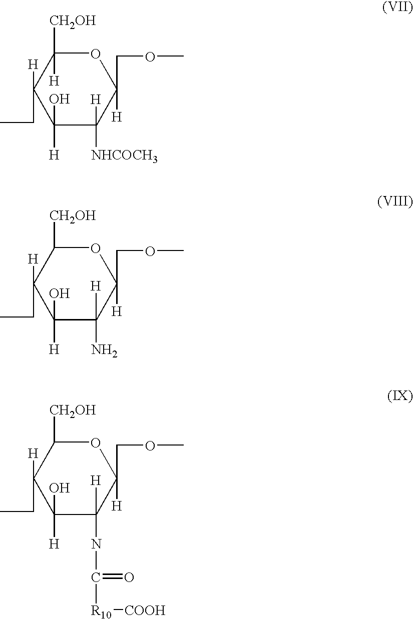
wherein the unit (VII) is present in an amount ranging from 0% to 30%, the unit (VIII) is present in an amount ranging from 5% to 50%, and the unit (IX) is present in an amount ranging from 30% to 90%, it being understood that, in the unit (IX), R₁₀ is chosen from radicals of formula: 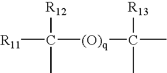
wherein

if q is equal to 0, then R₁₁, R₁₂ and R₁₃, which may be identical or different, are chosen from hydrogen atoms, and methyl, hydroxyl, acetoxy and amino radicals, monoalkylamine radicals and dialkylamine radicals that are optionally interrupted by at least one nitrogen atom and/or optionally substituted with at least one entity chosen from amine, hydroxyl, carboxyl, alkylthio and sulphonic groups, and alkylthio radicals in which the alkyl group bears an amino residue, wherein at least one of the radicals R₁₁, R₁₂ and R₁₃ being, in this case, a hydrogen atom;

or, if q is equal to 1, then R₁₁, R₁₂ and R₁₃ are each a hydrogen atom, and also the salts formed by these compounds with bases or acids.

(6) Polymers derived from the N-carboxyalkylation of chitosan, such as N-carboxymethylchitosan or N-carboxybutylchitosan.

(7) Polymers chosen from those of formula (X) such as those described, for example, in French Patent No. 1,400,366: 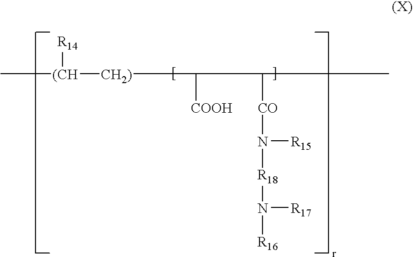
wherein R₁₄ is chosen from hydrogen atoms, and CH₃O, CH₃CH₂O and phenyl radicals, R₁₅ is chosen from a hydrogen atom and lower alkyl radicals, such as methyl or ethyl, R₁₆ is chosen from a hydrogen atom and lower alkyl radicals, such as methyl or ethyl, R₁₇ is chosen from lower alkyl radicals, such as methyl or ethyl, and radicals of formula: —R₁₈—N(R₁₆)₂, wherein R₁₈ is chosen from —CH₂—CH₂—, —CH₂—CH₂—CH₂— and —CH₂—CH(CH₃)— groups, and R₁₆ is chosen from a hydrogen atom and lower alkyl radicals, such as methyl or ethyl, and also the higher homologues of these radicals and comprising up to 6 carbon atoms, and wherein r is an integer greater than 1.

(8) Amphoteric polymers of formula -D-X-D-X- chosen from:

a) polymers obtained by the action of chloroacetic acid or sodium chloroacetate on compounds comprising at least one unit of formula (XI):
-D-X-D-X-D- (XI)
an oxygen atom and comprises at least one functional group chosen from carboxyl and hydroxyl functional groupss and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also comprise other vinyl comonomers such as vinylcaprolactam.

In one embodiment of the present disclosure, the at least one amphoteric polymer is chosen from those of family (1).

According to the present disclosure, the at least one cationic and/or amphoteric polymer can be present in an amount ranging from 0.001% to 20% by weight, for instance, from 0.01% to 10% by weight, such as from 0.02% to 5% by weight, relative to the total weight of the final composition.

The weight ratio of the cationic polymer to the high molecular weight water-soluble polymer can range from 100 to 0.0005, such as from 20 to 0.01, and for instance, from 1 to 0.01.

Silicones

According to another aspect of the present disclosure, the compositions may also comprise at least one silicone. Among the silicones that may be used in the compositions of the present disclosure, non-limiting mention may be made of, for example, volatile or non-volatile, cyclic or acyclic, branched or unbranched, organomodified or non-organomodified silicones, as described below.

where D is a radical

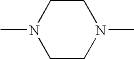

and X is chosen from E or E′, wherein E or E′, which may be identical or different, are divalent radicals chosen from straight or branched alkylene radicals comprising up to 7 carbon atoms in the main chain, which may be unsubstituted or substituted with hydroxyl groups and may optionally comprise at least one heteroatom chosen from oxygen, nitrogen and sulphur atoms, and optionally comprise 1 to 3 aromatic and/or heterocyclic rings; the oxygen, nitrogen and sulphur atoms being present in the form of ether, thioether, sulphoxide, sulphone, sulphonium, alkylamine, alkenylamine, hydroxyl, benzylamine, amine oxide, quaternary ammonium, amide, imide, alcohol, ester and/or urethane groups;

b) polymers of formula (XII):
-D-X-D-X- (XII)

where D is a radical

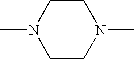

and X is chosen from E and E′, and at least once E′; wherein E has the meaning given above and E′ is a divalent radical chosen from straight and branched alkylene radicals comprising up to 7 carbon atoms in the main chain, which may be unsubstituted or substituted with at least one hydroxyl radicals, and comprising at least one nitrogen atom, wherein the nitrogen atom is substituted with an alkyl chain that is optionally interrupted by

The silicones that may be used as disclosed herein can be soluble or insoluble in the composition and, for instance, can be polyorganosiloxanes that are insoluble in the composition of the present disclosure; they may be in the form of oils, waxes, resins or gums.

According to the present disclosure, all the silicones may be used in unmodified form or in the form of solutions, dispersions, emulsions, nanoemulsions or microemulsions.

The organopolysiloxanes are defined in greater detail in Walter Noll's “Chemistry and Technology of Silicones” (1968) Academic Press. They can be volatile or non-volatile. When they are volatile, the silicones may be, for example, chosen from those having a boiling point ranging from 60° C. to 260° C. Non-limiting examples include:

(i) cyclic silicones comprising from 3 to 7, such as 4 to 5 silicon atoms. These are, for example, octamethylcyclotetrasiloxane sold, for instance, under the name “Volatile Silicone 7207” by Union Carbide or “Silbione 70045 V 2” by Rhodia, decamethylcyclopentasiloxane sold under the name “Volatile Silicone 7158” by Union Carbide, and “Silbione 70045 V 5” by Rhodia, and mixtures thereof. Non-limiting mention may also be made of cyclocopolymers of the dimethylsiloxane/methylalkylsiloxane type, such as “Volatile Silicone FZ 3109” sold by the company Union Carbide, having the chemical structure:

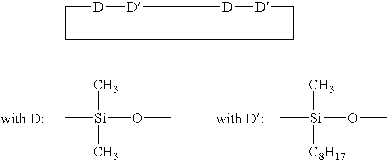

Further non-limiting mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones comprising 2 to 9 silicon atoms and having a viscosity of less than or equal to 5×10⁻⁶ m²/s at 25° C. An example is decamethyltetrasiloxane sold, for instance under the name “SH 200” by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics.”

Among the non-volatile silicones that may be used as disclosed herein, non-limiting mention may be made of polyalkylsiloxanes, polyarylsiloxanes, polyalkylarylsiloxanes, silicone gums, silicone resins, and polyorganosiloxanes modified with organofunctional groups, and also mixtures thereof. For example, these silicones may be chosen from polyalkylsiloxanes, among which non-limiting mention may be made of polydimethylsiloxanes comprising trimethylsilyl end groups and having a viscosity of from 5×10⁻⁶ to 2.5 m²/s at 25° C., and for instance 1×10⁻⁵ to 1 m²/s. The viscosity of the silicones is measured, for example, at 25° C. according to ASTM standard 445 Appendix C.

Among these polyalkylsiloxanes, mention may be made, in a non-limiting manner, of the following commercial products:

the Silbione® oils of the 47 and 70 047 series or the Mirasil® oils sold by Rhodia, such as, for example, the oil 70 047 V 500 000;

the oils of the Mirasil® series sold by the company Rhodia;

the oils of the 200 series from the company Dow Corning, such as, for example, DC200 with a viscosity of 60 000 mm²/s;

the Viscasil® oils from General Electric and certain oils of the SF series (SF 96, SF 18) from General Electric.

Non-limiting mention may also be made of polydimethylsiloxanes comprising dimethylsilanol end groups, known under the name dimethiconol (CTFA) such as the oils of the 48 series from the company Rhodia. In this category of polyalkylsiloxanes, non-limiting mention may also be made of the products sold under the names “Abil Wax® 9800 and 9801” by the company Goldschmidt, which are poly(C₁-C₂₀)alkylsiloxanes. The polyalkylarylsiloxanes may also be chosen from, for example, polydimethyl/methylphenylsiloxanes, linear and/or branched polydimethyidiphenylsiloxanes with a viscosity ranging from 1×10⁻⁵ to 5×10⁻² m²/s at 25° C.

Among these polyalkylarylsiloxanes, examples that may be mentioned in a non-limiting manner include the products sold under the following names:

the Silbione® oils of the series 70 641 from Rhodia;

the oils of the Rhodorsil® 70 633 and 763 series from Rhodia;

the oil Dow Corning 556 Cosmetic Grade Fluid from Dow Corning;

the silicones of the PK series from Bayer, for instance the product PK20;

the silicones of the PN and PH series from Bayer, for instance the products PN1000 and PH1000;
certain oils of the SF series from General Electric, such as SF 1023, SF 1154, SF 1250 and SF 1265.

The silicone gums that may be used in accordance with the present disclosure include, for example, polydiorganosiloxanes having high number-average molecular masses ranging from 200,000 to 1,000,000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Non-limiting mention may be made, for example, of the following products:

polydimethylsiloxane,

polydimethylsiloxane/methylvinylsiloxane gums,

polydimethylsiloxane/diphenylsiloxane,

polydimethylsiloxane/phenylmethylsiloxane, and

polydimethylsiloxane/diphenylsiloxane/methylvinylsiloxane.

Additional products that can be used as disclosed herein, include, for example, mixtures such as:

mixtures formed from a polydimethylsiloxane hydroxylated at the chain end, or dimethiconol (CTFA), and from a cyclic polydimethylsiloxane, also known as cyclomethicone (CTFA), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500,000, dissolved in the oil SF 1202 Silicone Fluid corresponding to decamethylcyclopentasiloxane;

mixtures of two PDMSs of different viscosities, for instance, a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product may comprise, for example, 15% SE 30 gum and 85% SF 96 oil.

The organopolysiloxane resins that can be used as disclosed herein are crosslinked siloxane systems comprising the units: R₂SiO_{2/2}, R₃SiO_{1/2}, RSiO_{3/2} and SiO_{4/2}, wherein R is chosen from hydrocarbon-based groups comprising from 1 to 16 carbon atoms and phenyl groups. For example, among these products, R may be chosen from C₁-C₄ lower alkyl groups, such as methyl and phenyl groups.

Among these resins, non-limiting mention may be made of the product sold under the name “Dow Corning 593” or those sold under the names “Silicone Fluid SS 4230 and SS 4267” by the company General Electric, which are silicones of dimethyl/trimethyl siloxane structure. Non-limiting mention may also be made of the trimethyl siloxysilicate type resins sold, for example, under the names X22-4914, X21-5034 and X21-5037 by the company Shin-Etsu.

The organomodified silicones that can be used in accordance with the present disclosure are silicones as defined above and comprising in their structure at least one organofunctional group attached via a hydrocarbon-based group. Among the organomodified silicones that may be used, non-limiting mention may be made of polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 or the oils Silwet® L 722, L 7500, L 77 and L 711 by the company Union Carbide, and the (C₁₂)alkylmethicone copolyol sold by the company Dow Corning-under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups can be, for example, C₁-C₄ aminoalkyl groups;

thiol groups such as the products sold under the names “GP 72 A” and “GP 71” from Genesee;

alkoxylated groups such as the product sold under the name “Silicone Copolymer F-755” by SWS Silicones and Abil Wax® 2428, 2434 and 2440 by the company Goldschmidt;

hydroxylated groups such as the polyorganosiloxanes comprising a hydroxyalkyl functional group, described in French Patent Application No. FR-A-85/16334;

acyloxyalkyl groups such as, for example, the polyorganosiloxanes described in U.S. Pat. No. 4,957,732;

anionic groups of carboxylic type, such as, for example, in the products described in European Patent No. EP 186 507 from the company Chisso Corporation, or of alkylcarboxylic type, such as those present in the product X-22-3701 E from the company Shin-Etsu; 2-hydroxyalkyl sulphonate; 2-hydroxyalkyl thiosulphate such as the products sold by the company Goldschmidt under the names “Abil® S201” and “Abil® S255”;

hydroxyacylamino groups, such as the polyorganosiloxanes described in European Patent Application No. EP 342 834. Non-limiting mention may be made, for example, of the product Q2-8413 from the company Dow Corning.

The at least one silicone can be present in a total amount ranging from 0.01% to 20% by weight, such as ranging from 0.1% to 5% by weight, relative to the total weight of the composition.

The composition according to the present disclosure may comprise at least one plant oil such as sweet almond oil, avocado oil, castor oil, olive oil, jojoba oil, sunflower oil, wheatgerm oil, sesame oil, groundnut oil, grapeseed oil, soybean oil, rapeseed oil, safflower oil, copra oil, corn oil, hazelnut oil, karite butter, palm oil, apricot kernel oil, calophyllum oil and mixtures thereof.

As used herein, the term “cosmetically acceptable medium” is understood to mean a medium that is compatible with keratin materials, for example such as the skin, the eyelashes and the hair.

The cosmetically acceptable medium may comprise only water or comprise a mixture of water and at least one cosmetically acceptable solvent such as a C₁-C₄ lower alcohol, for instance ethanol, isopropanol, tert-butanol or n-butanol; alkylene glycols, for instance propylene glycol, and polyol ethers and mixtures thereof.

For example, the composition can comprise from 50% to 95% by weight of water relative to the total weight of the composition.

The washing compositions according to the present disclosure have a final pH ranging from 3 to 10, for instance, from 4.5 to 8. The pH may be adjusted to the desired value conventionally, by adding a base (organic or mineral base) to the composition, for example aqueous ammonia or a primary, secondary or tertiary (poly)amine, for instance monoethanolamine, diethanolamine, triethanolamine, isopropanolamine or 1,3-propanediamine, or alternatively by adding an acid, such as a carboxylic acid, for instance citric acid.

The compositions in accordance with the present disclosure may further comprise, in addition to the combination defined above, viscosity regulators such as electrolytes, or thickeners (associative or non-associative thickeners). Non-limiting mention may be made, for example, of sodium chloride, sodium xylenesulphonate, scleroglucans, xanthan gums, fatty acid alkanolamides, alkyl ether carboxylic acid alkanolamides optionally oxyethylenated with up to 5 mol of ethylene oxide, such as the product sold under the name “Aminol A15” by the company Chem Y, crosslinked polyacrylic acids and acrylic acid copolymers such as crosslinked acrylic acid/C₁₀-C₃₀ alkyl acrylate copolymers. The at least one viscosity regulator can be present in the compositions as disclosed herein in an amount ranging from 0% to 10% by weight, relative to the total weight of the composition.

The compositions in accordance with the present disclosure may also comprise up to 5% of at least one nacreous agent and/or opacifier that are well known in the art, such as, for example, fatty alcohols higher than C16, fatty-chain acyl derivatives such as ethylene glycol or polyethylene glycol monostearates or distearates, and fatty-chain (C₁₀-C₃₀) ethers such as, for example, distearyl ether or 1-(hexadecyloxy)-2-octadecanol.

The compositions as disclosed herein may optionally also comprise at least one additive chosen from foam synergists such as C₁₀-C₁₈ 1,2-alkanediols and fatty alkanolamides derived from monoethanolamine or diethanolamine, silicone and non-silicone sunscreens, anionic and nonionic polymers, cationic surfactants, proteins, protein hydrolysates, ceramides, pseudoceramides, linear and branched C₁₂-C₄₀ fatty acids such as 18-methyleicosanoic acid, hydroxy acids, vitamins, provitamins such as panthenol, animal, mineral and synthetic oils and any other additive conventionally used in cosmetics that does not affect the properties of the compositions according to the present disclosure.

Needless to say, the washing compositions as disclosed herein may also comprise at least one adjuvant usually encountered in the field of shampoos, for instance fragrances, preserving agents, sequestering agents, softeners, dyes, moisturizers, anti-dandruff agents or anti-seborrhoeic agents, and the like.

Of course, a person skilled in the art will take care to select any optional additional compound(s) and/or the amounts thereof such that the beneficial properties intrinsically associated with the combination in accordance with the present disclosure are not, or are not substantially, adversely affected by the envisioned addition(s).

The compositions as disclosed herein may be in the form of optionally thickened liquids, creams or gels, and they are suitable for washing, optionally caring for and/or styling the hair.

The present disclosure also relates to a cosmetic process for treating keratin materials, comprising applying an effective amount of a composition as described above to the keratin materials, and optionallyrinsing after an optional leave-in time.

In one embodiment of the present disclosure, the composition is used as a shampoo. When the compositions as disclosed herein are used as standard shampoos, they can be simply applied to wet hair and the lather generated by massaging or friction with the hands is then removed, after an optional action time, by rinsing with water, the operation possibly being repeated at least one time.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contain certain errors necessarily resulting from the standard deviation found in their respective testing measurements.

The following examples are intended to illustrate the invention in a non-limiting manner.

### EXAMPLES 1 to 4

Shampoo compositions in accordance with the present disclosure were prepared:

| Constituent | 1 | 2 | 3 | 4 |
|---|---|---|---|---|
| Copolymer (90 mol/10 mol) of | 1% AM | — | 0.75% AM | 0.5% AM |
| acrylamide and of dimethylaminoethyl | | | | |
| acrylate quaternized with | | | | |
| benzyl chloride (MW > 5 × 10⁶) as a | | | | |
| dispersion in a concentrated saline | | | | |
| aqueous solution (Ultimer from | | | | |
| Ondeo) | | | | |
| Copolymer (10 mol/90 mol) of | — | 0.5% AM | — | — |
| dimethylaminoethyl acrylate | | | | |
| quaternized with methyl chloride | | | | |
| and of acrylamide (MW > 5 × 10⁶) | | | | |
| as a dispersion in a concentrated | | | | |
| saline aqueous solution | | | | |
| Sodium lauryl ether sulphate with | 12.5% AM | — | 7.5% AM | 10% AM |
| 2.2 mol of ethylene oxide at 26% | | | | |
| AM | | | | |
| Ammonium lauryl ether sulphate 2 | — | 12.5% AM | — | — |
| EO | | | | |
| Cocoamidopropylbetaine | 2.5% AM | 2.5% AM | 2.5% AM | — |
| Disodium cocoamphodiacetate | — | — | — | 2% AM |
| Polyquaternium-10 (Celquat SC | 0.2% | — | — | — |
| 240 C from ICI) | | | | |
| Chitosan PCA (Kytamer PC from | — | 0.1% | 0.05% | — |
| AMERCHOL) | | | | |
| Polyquaternium-6 (Merquat 100 | — | — | — | 0.1% |
| from NALCO) | | | | |
| Ethanol | — | 1% | — | — |
| NaCl | — | — | — | 2% |
| Citric acid or NaOH qs | pH 6.5 | pH 6.5 | pH 6.5 | pH 6.5 |
| Water qs | 100% | 100% | 100% | 100% |

Hair treated with these shampoos had good styling and volumizing properties.

What is claimed is:

1. A composition for washing keratin materials, comprising, in a cosmetically acceptable aqueous medium,
at least one polymer chosen from cationic and amphoteric polymers,
at least one detergent surfactant chosen from anionic, nonionic and amphoteric detergent surfactants, and
at least one drawing polymer with a drawing power of greater than 5 cm.

2. The composition according to claim 1, wherein the at least one detergent surfactant is chosen from anionic surfactants.

3. The composition according to claim 1, wherein the at least one detergent surfactant is present in an amount ranging from 4% to 50% by weight, relative to the total weight of the composition.

4. The composition according to claim 3, wherein the at least one detergent surfactant is present in an amount ranging from 8% to 25% by weight, relative to the total weight of the composition.

5. The composition according to claims 1, wherein the at least one drawing polymer is chosen from either:
(a1) a dispersion of particles of at least one water-soluble polymer with a weight-average molecular mass of greater than 10⁶ in a saline aqueous solution, obtained by heterogeneous free-radical polymerization of water-soluble monomers with precipitation of the polymer formed, wherein at least one of the monomers is cationic, or
(a2) an aqueous saline solution of at least one water-soluble polymer with a weight-average molecular mass of greater than 10⁶, obtained by heterogeneous free-radical polymerization of water-soluble monomers with precipitation of the polymer formed, wherein at least one of the monomers is cationic.

6. The composition according to claim 5, wherein the water-soluble monomers are chosen from cationic, anionic and nonionic monomers comprising at least one ethylenic double bond, or a mixture thereof, wherein at least one of the monomers is cationic.

7. The composition according to claim 6, wherein the anionic monomers are chosen from acrylic acid, methacrylic acid, acrylamido-2-methylpropanesulphonic acid and itaconic acid.

8. The composition according to claim 6, wherein the nonionic monomers are chosen from acrylamide, methacrylamide, N-vinylformamide, N-vinylacetonamide, hydroxypropyl acrylate and hydroxypropyl methacrylate.

9. The composition according to claim 6, wherein the cationic monomers are chosen from di(C₁₋₄ alkyl)diallylammonium salts and the compounds of formula (I) 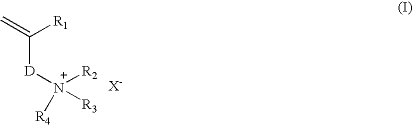
wherein:
R₁ is chosen from a hydrogen atom and methyl groups,
R₂ and R₃, which may be identical or different, are chosen from hydrogen atoms and linear and branched C₁₋₄ alkyl groups,
R₄ is chosen from a hydrogen atom, linear and branched C₁₋₄ alkyl groups and aryl groups,
D is chosen from the following unit 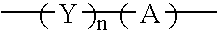
wherein
Y is chosen from amide, ester, urethane and urea functional groups,
A is chosen from linear, branched and cyclic C₁₋₁₀ alkylene group, which may be substituted or interrupted with a divalent aromatic or heteroaromatic ring, or which may be interrupted with a hetero atom chosen from O, N, S and P, and which may comprise a ketone, amide, ester, urethane or urea functional group,
n is equal to 0 or 1, and
X⁻ is an anionic counterion.

10. The composition according to claim 5, wherein the at least one water-soluble drawing polymer is obtained by heterogeneous free-radical polymerization of at least one cationic monomer of formula (I).

11. The composition according to claim 10, wherein the at least one water-soluble drawing polymer is obtained by heterogeneous free-radical polymerization of a monomer mixture comprising from 0 mol % to 30 mol % of acrylic acid, from 0 mol % to 95.5 mol % of acrylamide, and from 0.5 mol % to 100 mol % of at least one cationic monomer of formula (I).

12. The composition according to claim 11, wherein the at least one water-soluble drawing polymer is obtained by heterogeneous free-radical polymerization of a monomer mixture comprising acrylic acid and a cationic monomer of formula (I), wherein the number of moles of the cationic monomer of formula (I) is greater than the number of moles of acrylic acid.

13. The composition according to claim 11, wherein the at least one water-soluble drawing polymer is obtained by heterogeneous free-radical polymerization of a monomer mixture comprising 10 mol % of acryloyloxyethyldimethylbenzylammonium chloride and 90 mol % of acrylamide.

14. The composition according to claim 11, wherein the at least one water-soluble drawing polymer is obtained by heterogeneous free-radical polymerization of a monomer mixture comprising 30 mol % of acryloyloxytrimethylammonium chloride, 50 mol % of acryloyloxyethyldimethylbenzylammonium chloride and 20 mol % of acrylamide.

15. The composition according to claim 11, wherein the at least one water-soluble drawing polymer is obtained by heterogeneous free-radical polymerization of 10 mol % of acryloyloxyethyltrimethylammonium chloride and 90 mol % of acrylamide.

16. The composition according to claim 11, wherein the at least one water-soluble drawing polymer is obtained by heterogeneous free-radical polymerization of 30 mol % of diallyldimethylammonium chloride and 70 mol % of acrylamide.

17. The composition according to claim 5, wherein the at least one water-soluble drawing polymer whose particles are present as a dispersion in a saline aqueous solution in (a1) is present in an amount ranging from 0.01% to 20% by weight, relative to the total weight of the dispersion.

18. The composition according to claim 5, wherein the at least one water-soluble drawing polymer present as a saline aqueous solution in (a2) is present in the composition in an amount ranging from 0.01% to 20% by weight, relative to the total weight of the solution.

19. The composition according to claim 5, wherein the saline aqueous solution in (a1) or (a2) comprises at least one anionic salt.

20. The composition according to claim 19, wherein the at least one anionic salt is chosen from ammonium sulphate, ammonium hydrogen sulphate, sodium sulphate, sodium hydrogen sulphate, magnesium sulphate, magnesium hydrogen sulphate, aluminium sulphate and aluminium hydrogen sulphate.

21. The composition according to claim 1, wherein the at least one drawing polymer having a drawing power of greater than 5 cm is present in the composition in an amount ranging from 0.01% to 10% by weight, relative to the total weight of the composition.

22. The composition according to claim 1, wherein the at least one cationic polymer is chosen from those comprising units comprising primary, secondary, tertiary and/or quaternary amine groups that either may form part of the main polymer chain or may be borne by a side substituent directly attached thereto.

23. The composition according to claim 1, wherein the at least one cationic polymer is chosen from:
(1) homopolymers and copolymers derived from acrylic and methacrylic esters and amides and comprising at least one unit of the following formulae: 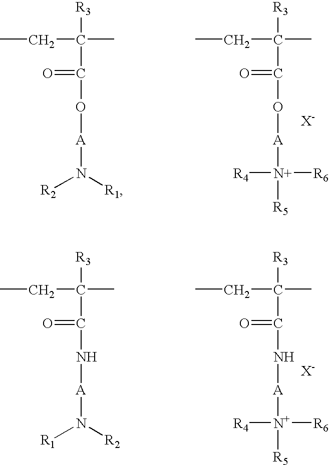
wherein:
R₃, which may be identical or different, is chosen from a hydrogen atom and CH₃ radicals;
A, which may be identical or different, is chosen from linear and branched alkyl groups comprising from 1 to 6 carbon atoms and hydroxyalkyl groups comprising from 1 to 4 carbon atoms;
R₄, R₅ and R₆, which may be identical or different, are chosen from alkyl groups comprising from 1 to 18 carbon atoms and benzyl radicals;
R₁ and R₂, which may be identical or different, are chosen from hydrogen atoms and alkyl groups comprising from 1 to 6 carbon atoms;
X is an anion derived from a mineral or organic acid;
(2) cationic polysaccharides,
(3) polymers comprising piperazinyl units and divalent alkylene and hydroxyalkylene radicals comprising straight and branched chains, optionally interrupted by oxygen, sulphur or nitrogen atoms or by aromatic or heterocyclic rings, and the oxidation and/or quaternization products of said polymers,
(4) water-soluble polyamino amides prepared by polycondensation of an acidic compound with a polyamine, wherein the polyamino amides can optionally be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound that is reactive with respect to a bis-halohydrin, a bis-azetidinium, a bis-haloacyldiamine, a bis-alkyl halide, an epihalohydrin, a diepoxide or a bis-unsaturated derivative; the crosslinking agent being used in an amount ranging from 0.025 mol to 0.35 mol per amine group of the polyamino amide; and wherein the polyamino amides are optionally alkylated or, if they comprise at least one tertiary amine functional group, optionally quaternized;
(5) polyamino amide derivatives resulting from the condensation of polyalkylene polyamines with polycarboxylic acids followed by alkylation with difunctional agents,
(6) polymers obtained by reaction of a polyalkylene polyamine comprising two primary amine groups and at least one secondary amine group with a dicarboxylic acid chosen from diglycolic acid and saturated aliphatic dicarboxylic acids comprising from 3 to 8 carbon atoms,
(7) cyclopolymers of alkyldiallylamine or of dialkyldiallylammonium,
(8) diquaternary ammonium polymers comprising repeating units of formula (II): 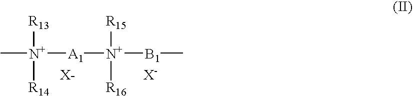
wherein:
R₁₃, R₁₄, R₁₅ and R₁₆, which may be identical or different, are chosen from aliphatic, alicyclic and arylaliphatic radicals comprising from 1 to 20 carbon atoms and lower hydroxyalkylaliphatic radicals, or alternatively R₁₃, R₁₄, R₁₅ and R₁₆, together or separately, can form, with the nitrogen atoms to which they are attached, heterocycles optionally comprising a second hetero atom other than nitrogen, or alternatively R₁₃, R₁₄, R₁₅ and R₁₆ are chosen from linear and branched C₁-C₆ alkyl radicals substituted with a nitrile, ester, acyl or amide group or a group —CO—O—R₁₇-D or —CO—NH—R₁₇-D where R₁₇ is an alkylene and D is a quaternary ammonium group;
A₁ and B₁ are chosen from linear and branched, and saturated and unsaturated polymethylene groups comprising from 2 to 20 carbon atoms, which may comprise, linked to or intercalated in the main chain, at least one aromatic ring or at least one entity chosen from oxygen and sulphur atoms and sulphoxide, sulphone, disulphide, amino, alkylamino, hydroxyl, quaternary ammonium, ureido, amide and ester groups, and
X⁻ is an anion derived from a mineral or organic acid;
A₁, R₁₃ and R₁₅ can form, with the two nitrogen atoms to which they are attached, a piperazine ring; and if A₁ is chosen from linear and branched, saturated and unsaturated alkylene and hydroxyalkylene radicals, then B₁ can also be chosen from (CH₂)ₙ—CO-D-OC—(CH₂)ₙ—groups
wherein D is chosen from:
a) glycol groups of formula: —O-Z-O—, where Z is chosen from linear and branched hydrocarbon-based radicals and groups of formulae: —(CH₂—CH₂—O)ₓ—CH₂—CH₂—, and —[CH₂—CH(CH₃)—O]_{y}—CH₂—CH(CH₃)—
where x and y are an integer from 1 to 4, representing a defined and unique degree of polymerization or any number from 1 to 4 representing an average degree of polymerization;
b) bis-secondary diamine groups;
c) bis-primary diamine groups of formula: —NH—Y—NH—, where Y is chosen from linear and branched hydrocarbon-based radicals, or alternatively the divalent radical —CH₂—CH₂—S—S—CH₂—CH₂—;
d) ureylene groups of formula: —NH—CO—NH—;
(9) polyquaternary ammonium polymers comprising units of formula (III): 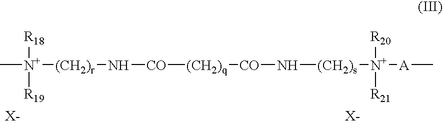
wherein:
R₁₈, R₁₉, R₂₀ and R₂₁, which may be identical or different, are chosen from hydrogen atoms and methyl, ethyl, propyl, β-hydroxyethyl, β-hydroxypropyl and —CH₂CH₂(OCH₂CH₂)ₚOH radicals,
p is an integer ranging from 0 to 6, with the proviso that R₁₈, R₁₉, R₂₀ and R₂₁ are not all simultaneously hydrogen atoms,
r and s, which may be identical or different, are integers ranging from 1 to 6,
q is an integer ranging from 0 to 34,
X is chosen from halogen atoms, and
A is chosen from dihalide radicals and —CH₂—CH₂—O—CH₂—CH₂— groups;
(10) quaternary polymers of vinylpyrrolidone and of vinylimidazole,
(11) crosslinked polymers of methacryloyloxy(C₁-C₄)alkyltri(C₁-C₄)alkylammonium salts, and
(12) polyalkyleneimines.

24. The composition according to claim 23, wherein the at least one cationic polymer is chosen from cationic cyclopolymers, quaternary polymers of vinylpyrrolidone and of vinylimidazole, crosslinked homopolymers and copolymers of methacryloyloxyalkyl(C₁-C₄)trialkyl(C₁-C₄)ammonium salts, and chitosan pyrrolidonecarboxylate.

25. The composition according to claim 24, wherein the cyclopolymers are chosen from homopolymers of diallyldimethylammonium chloride and copolymers of diallyldimethylammonium chloride and acrylamide.

26. The composition according to claim 1, wherein the at least one amphoteric polymer is chosen from:
(1) polymers resulting from the copolymerization of a monomer derived from a vinyl compound bearing a carboxylic group, and a basic monomer derived from a substituted vinyl compound comprising at least one basic atom, and salts thereof,
(2) polymers comprising units derived from:
a) at least one monomer chosen from acrylamides and methacrylamides substituted on the nitrogen with an alkyl radical,
b) at least one acidic comonomer comprising at least one reactive carboxylic group, and
c) at least one basic comonomer comprising primary, secondary, tertiary and quaternary amine substituents of acrylic and methacrylic acids and the product of quaternization of dimethylaminoethyl methacrylate with dimethyl or diethyl sulphate;
(3) Polyamino amides that are crosslinked and alkylated partially or totally derived from polyamino amides of formula (IV): 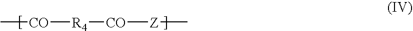
wherein R₄ is chosen from divalent radicals derived from saturated dicarboxylic acids, mono- and dicarboxylic aliphatic acids comprising an ethylenic double bond, esters of a lower alkanol comprising from 1 to 6 carbon atoms of these acids and radicals derived from the addition of any one of the acids to bis(primary) and bis(secondary) amines, and Z is chosen from bis(primary), mono- and bis(secondary) polyalkylene-polyamine radicals,
(4) Polymers comprising zwitterionic units of formula (VI): 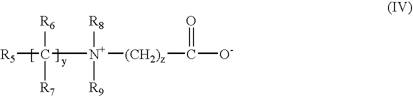
wherein R₅ is chosen from polymerizable unsaturated groups, y and z are integers from 1 to 3, R₆ and R₇ are chosen from hydrogen atoms, methyl, ethyl and propyl groups, R₈ and R₉ are chosen from hydrogen atoms and alkyl radicals such that the sum of the carbon atoms in R₈ and R₉ does not exceed 10;
(5) polymers derived from chitosan comprising monomer units of formulae (VII), (VII) and (IX): 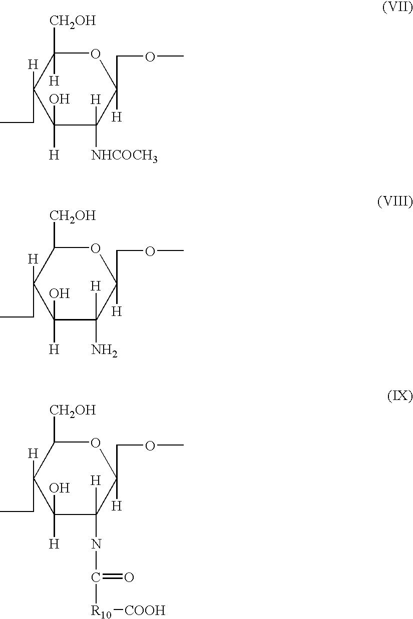
wherein the unit (VII) is present in an amount ranging from 0% to 30%, the unit (VIII) is present in an amount ranging from 5% to 50%, and the unit (IX) is present in an amount ranging from 30% to 90%, wherein, in the unit (IX), R₁₀ is chosen from groups of formula: 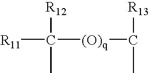
wherein
if q is equal to 0, then R₁₁, R₁₂ and R₁₃, which may be identical or different, are chosen from hydrogen atoms, and methyl, hydroxyl, acetoxy and amino groups, and monoalkylamine and dialkylamine groups that are optionally interrupted by at least one nitrogen atom and/or optionally substituted with at least one groups chosen from amine, hydroxyl, carboxyl, alkylthio and sulphonic groups, and alkylthio groups in which the alkyl group bears an amino residue, at least one of the radicals R₁₁, R₁₂ and R₁₃ being, in this case, a hydrogen atom;
or, if q is equal to 1, then R₁₁, R₁₂ and R₁₃ are all hydrogen atoms, and also the salts formed by these compounds with bases or acids,
(6) polymers derived from the N-carboxyalkylation of chitosan,
(7) polymers of formula (X): 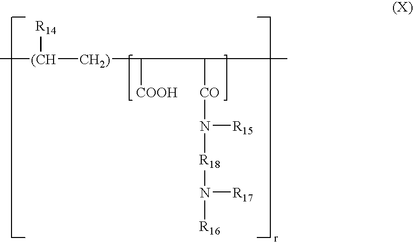
wherein R₁₄ is chosen from a hydrogen atom, and CH₃O, CH₃CH₂O and phenyl radicals, R₁₅ is chosen from a hydrogen atom and lower alkyl radicals, R₁₆ is chosen from a hydrogen atom or lower alkyl radicals, R₁₇ is chosen from lower alkyl radicals and radicals of formula: —R₁₈—N(R₁₆)₂, wherein R₁₈ is chosen from —CH₂—CH₂—, —CH₂—CH₂—CH₂— and —CH₂—CH(CH₃)— groups, and R₁₆ is chosen from a hydrogen atom or lower alkyl radicals, and also the higher homologues of these radicals and comprising up to 6 carbon atoms, and wherein r is an integer greater than 1,
(8) Amphoteric polymers of -D-X-D-X- chosen from:
a) polymers obtained by the action of chloroacetic acid or sodium chloroacetate on compounds comprising at least one unit of formula:
-D-X-D-X-D- (XI)
where D is the radical 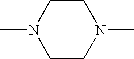
and X is chosen from E and E′, wherein E or E′, which may be identical or different, are divalent radicals chosen from straight and branched chain alkylene radicals comprising up to 7 carbon atoms in the main chain, which is unsubstituted or substituted with hydroxyl groups, and may optionally comprise at least one heteroatom chosen from oxygen, nitrogen and sulphur atoms, and may optionally comprise 1 to 3 aromatic and/or heterocyclic rings; the oxygen, nitrogen and sulphur atoms being present in the form of ether, thioether, sulphoxide, sulphone, sulphonium, alkylamine, alkenylamine, hydroxyl, benzylamine, amine oxide, quaternary ammonium, amide, imide, alcohol, ester and/or urethane groups;
b) polymers of formula:
-D-X-D-X- (XII)
where D is the radical 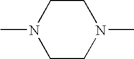
and X is chosen from E and E′, and at least once E′; wherein E is a divalent radical chosen from straight and branched chain alkylene radicals comprising up to 7 carbon atoms in the main chain, which is unsubstituted or substituted with hydroxyl groups, and may optionally comprise at least one heteroatom chosen from oxygen, nitrogen and sulphur atoms, and may optionally comprise 1 to 3 aromatic and/or heterocyclic rings; the oxygen, nitrogen and sulphur atoms being present in the form of ether, thioether, sulphoxide, sulphone, sulphonium, alkylamine, alkenylamine, hydroxyl, benzylamine, amine oxide, quaternary ammonium, amide, imide, alcohol, ester and/or urethane groups, and E′ is a divalent radical that is chosen from straight and branched chain alkylene radicals comprising up to 7 carbon atoms in the main chain, which is unsubstituted or substituted with at least one hydroxyl radical and comprising at least one nitrogen atom, the nitrogen atom being substituted with an alkyl chain that is optionally interrupted by an oxygen atom and comprises at least one functional group chosen from carboxyl and hydroxyl functional groups and betainized by reaction with chloroacetic acid or sodium chloroacetate, and
(9) (C₁-C₅)alkyl vinyl ether/maleic anhydride copolymers partially modified by semiamidation with an N,N-dialkylaminoalkylamine or by semiesterification with an N,N-dialkanolamine.

27. The composition according to claim 26, wherein the at least one amphoteric polymer is chosen from the copolymers of dimethyldiallylammonium salt and acrylic acid.

28. The composition according to claim 1, wherein the at least one cationic and/or amphoteric polymer is present in a total amount ranging from 0.001% to 20% by weight, relative to the total weight of the final composition.

29. The composition according to claim 1, further comprising at least one silicone.

30. The composition according to claim 29, wherein the at least one silicone is present in an amount ranging from 0.01% to 20% by weight, relative to the total weight of the composition.

31. The composition according to claim 1, wherein the pH ranges from 3 to 10.

32. A process for treating keratin materials, comprising,
applying to the keratin materials an effective amount of a cosmetic composition comprising, in a cosmetically acceptable aqueous medium, at least one polymer chosen from cationic and amphoteric polymers, at least one detergent surfactant chosen from anionic, nonionic and amphoteric detergent surfactants, and at least one drawing polymer with a drawing power of greater than 5 cm, optionally followed by rinsing.

33. A process according to claim 32, where treating comprises at least one of cleaning, caring for, conditioning, and styling.
